(19) United States
(12) Patent Application Publication
(10) Pub. No.: US 2026/0069525 A1
(43) Pub. Date: Mar. 12, 2026

(54) ANTIMICROBIAL PEPTIDE FOR MAINTAINING HEALTH OF SCALP MICROBIOME AND METHOD FOR PREVENTION OR TREATMENT OF SCALP DISEASE USING THE SAME

(71) Applicant: Metanovas Biotech Inc., San Francisco, CA (US)

(72) Inventors: Lun YU, San Jose, CA (US); Heng LUO, Brooklyn, NY (US); Meijie WANG, Santa Clara, CA (US)

(21) Appl. No.: 19/263,466

(22) Filed: Jul. 8, 2025

Related U.S. Application Data
(63) Continuation-in-part of application No. PCT/IB2025/056310, filed on Jun. 20, 2025.

(30) Foreign Application Priority Data
Sep. 12, 2024 (CN) 202411281077.0

(51) Int. Cl. A61K 8/64; A61K 38/00; A61P 31/04; A61P 31/10; A61Q 5/00; C07K 7/08

(52) U.S. Cl. CPC A61K 8/64; A61P 31/04; A61P 31/10; A61Q 5/006; C07K 7/08; A61K 38/00

(57) ABSTRACT

An antimicrobial peptide for maintaining the health of scalp microbiome, including one of the following polypeptides (1)-(7): (1) a polypeptide including an amino acid sequence shown in SEQ ID NO: 1, or a polypeptide consisting of amino acid residues of SEQ ID NO: 1; (2) a polypeptide including an amino acid sequence shown in SEQ ID NO: 2, or a polypeptide consisting of amino acid residues of SEQ ID NO: 2; (3) a polypeptide resulting from substitution, deletion, and/or addition of one or more amino acid residues of the polypeptide in (1) or (2); (4) a polypeptide obtained by modifying a N-terminus and/or a C-terminus of the polypeptide in (1) or (2); and (5) a polypeptide having at least 85%, 90%, 95%, 98%, or 99% sequence identity to the amino acid sequence of the polypeptide in (1) or (2). The antimicrobial peptide exhibits excellent safety for treating scalp conditions.

### CROSS-REFERENCE TO RELATED APPLICATIONS

This application is a continuation-in-part of International Patent Application No. PCT/IB2025/056310 with an international filing date of Jun. 20, 2025, designating the United States, now pending, and further claims foreign priority benefits to Chinese Patent Application No. 202411281077.0 filed Sep. 12, 2024. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application comprises a sequence listing, which has been submitted electronically in XML file and is incorporated herein by reference in its entirety. The XML file, created on Apr. 8, 2025, is named SZWK-00301-UUS. xml, and is 16,772 bytes in size.

### BACKGROUND

The disclosure relates to the field of antimicrobial peptides (AMPs), and more particularly, to an antimicrobial peptide for maintaining the health of the scalp microbiome and a method for prevention or treatment of a scalp disease using the same.

The scalp is a unique ecological niche characterized by dense hair coverage, abundant sweat glands, sebaceous glands and high relative humidity, creating favorable conditions for microbial colonization and growth. Disruption of the scalp microenvironment can lead to an imbalance, manifesting as reduced microbiome diversity, structural shifts in microbial communities, and dysfunction in host skin barrier and metabolic activities. Such pathological changes can result in various scalp disorders and diseases, including dandruff, seborrheic dermatitis, scalp pruritus, and hair loss (alopecia).

Antimicrobial peptides (AMPs), as a new type of antibacterial agent, have a wide range of sources and broad-spectrum antibacterial activity. Compared with traditional antibiotics, most antimicrobial peptides primarily kill bacteria by disrupting their cell membranes, making it difficult for bacteria to develop resistance. Antimicrobial peptides are considered promising alternatives to antibiotics. Despite their significance, natural AMPs face limitations in practical applications due to high production costs, low antimicrobial activity, and poor stability. With advancing research on the properties, structures, and mechanisms of existing natural AMPs, increasing efforts are focused on the development of synthetic antimicrobial peptides. Currently, research on AMPs for maintaining the health of the scalp microbiome remains limited.

### SUMMARY

To address the aforementioned issues, the disclosure provides an antimicrobial peptide for maintaining the health of the scalp microbiome, the antimicrobial peptide comprising one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide resulting from substitution, deletion, and/or addition of one or more amino acid residues of the polypeptide in (1) or (2);
(4) a polypeptide obtained by modifying a N-terminus and/or a C-terminus of the polypeptide in (1) or (2); and
(5) a polypeptide having at least 85%, 90%, 95%, 98%, or 99% sequence identity to the amino acid sequence of the polypeptide in (1) or (2).
In a class of this embodiment, the antimicrobial peptide is selected from P16, P16L, P16K, P16R, P16I, or P16V.

In a class of this embodiment, the amino acid sequence of the antimicrobial peptide P16 is shown in SEQ ID NO: 1; the amino acid sequence of the antimicrobial peptide P16L is shown in SEQ ID NO: 2; and the amino acid sequence of the antimicrobial peptide P16K is shown in SEQ ID NO: 3.

In a class of this embodiment, the amino acid sequence of the antimicrobial peptide P16R is shown in SEQ ID NO: 4; the amino acid sequence of the antimicrobial peptide P16I is shown in SEQ ID NO: 5; and the amino acid sequence of the antimicrobial peptide P16V is shown in SEQ ID NO: 6.

In another aspect, the disclosure further provides a method for preparing the antimicrobial peptide; the method employs a solid-phase synthesis technique.

The disclosure also provides a method for prevention or treatment of a scalp disease caused by dysbiosis of scalp microbiome, the method comprising: administering to a patient in need thereof a product comprising a therapeutically effective amount of the antimicrobial peptide.

In a class of this embodiment, the scalp disease comprises dandruff, seborrheic dermatitis, pityriasis versicolor, and Malassezia folliculitis, all of which are associated with fungal infections.

In a class of this embodiment, the product is selected from the group consisting of scalp care products, hair conditioners, shampoos, and topical medications.

The antimicrobial peptide for maintaining the health of the scalp microbiome disclosed herein exhibits excellent safety for treating scalp conditions caused by microbial dysbiosis, including dandruff, seborrheic dermatitis, scalp pruritus, and hair loss. The antimicrobial peptide can be incorporated into anti-dandruff or therapeutic formulations such as scalp care products, conditioners, shampoos, or topical pharmaceuticals targeting seborrheic dermatitis, pruritus, or alopecia. Notably, the antimicrobial peptide demonstrates significant inhibitory effects against pathogenic microbes—Malassezia furfur, Malassezia restricta, Staphylococcus hominis, Candida albicans, and Staphylococcus aureus—while preserving the growth of beneficial bacteria (e.g., Lactobacillus acidophilus). This selective antimicrobial activity promotes scalp microbiome homeostasis, highlighting its substantial therapeutic and commercial potential.

### BRIEF DESCRIPTION OF THE DRAWINGS

FIGS. 1A and 1B illustrate the inhibition rates of the antimicrobial peptides (AFP) of the disclosure against Malassezia furfur ATCC 44344 after 48 and 72 hours of incubation of the bacteria with 10 μg/mL and 20 μg/mL of the antimicrobial peptides;

FIGS. 2A and 2B illustrate the inhibition rates of the antimicrobial peptides of the disclosure against Malassezia restricta after 48 and 72 hours of incubation of the bacteria with 10 μg/mL and 20 μg/mL of the antimicrobial peptides;

FIGS. 3A and 3B illustrate the inhibition rates of the antimicrobial peptides of the disclosure against Staphylococcus hominis after 48 and 72 hours of incubation of the bacteria with 10 μg/mL and 20 μg/mL of the antimicrobial peptides;

FIG. 4 illustrates the inhibition rates of the antimicrobial peptides of the disclosure against Candida albicans after 24 hours of incubation of the bacteria with 100 μg/mL of the antimicrobial peptides;

FIG. 5 illustrates the inhibition rates of the antimicrobial peptides of the disclosure against Staphylococcus aureus after 24 hours of incubation of the bacteria with 100 μg/mL of the antimicrobial peptides;

FIGS. 6A and 6B illustrate the inhibition rates of the antimicrobial peptides of the disclosure against Lactobacillus acidophilus after 48 and 72 hours of incubation of the bacteria with 10 μg/mL, 20 μg/mL, and 40 μg/mL of the antimicrobial peptides;

FIGS. 7A, 7B, and 7C illustrate the anti-inflammatory effects of the antimicrobial peptides of the disclosure after 24 hours of incubation in the murine macrophage cell line RAW264.7 with an addition of 20 μg/ml;

FIGS. 8A, 8B, and 8C illustrate the anti-allergy effects of the antimicrobial peptides of the disclosure after 24 hours of incubation in the murine mastocytoma cell line P815 with an addition of 20 μg/mL;

FIGS. 9A-9E illustrate the experimental results of the antimicrobial peptide of the disclosure after 28 days of application at a concentration of 0.2% in a human dandruff removal experiment; and

FIG. 10 illustrate the anti-fungal test results of the antimicrobial peptide P16L variants against Malassezia restricta.

### DETAILED DESCRIPTION

To further illustrate the disclosure, embodiments detailing an antimicrobial peptide for maintaining the health of the scalp microbiome and method for prevention or treatment of a scalp disease using the same are described below. It should be noted that the following embodiments are intended to describe and not to limit the disclosure.

Specifically, the disclosure provides an antimicrobial peptide, comprising one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide resulting from substitution, deletion, and/or addition of one or more amino acid residues of the polypeptide in (1) or (2);
(4) a polypeptide obtained by modifying a N-terminus and/or a C-terminus of the polypeptide in (1) or (2); and
(5) a polypeptide having at least 85%, 90%, 95%, 98%, or 99% sequence identity to the amino acid sequence of the polypeptide in (1) or (2).

Preferably, the antimicrobial peptide is selected from P16, P16L, P16K, P16R, P16I, or P16V.

The amino acid sequence of the antimicrobial peptide P16 is shown in SEQ ID NO: 1; the amino acid sequence of the antimicrobial peptide P16L is shown in SEQ ID NO: 2; the amino acid sequence of the antimicrobial peptide P16K is shown in SEQ ID NO: 3; the amino acid sequence of the antimicrobial peptide P16R is shown in SEQ ID NO: 4; the amino acid sequence of the antimicrobial peptide P16I is shown in SEQ ID NO: 5; and the amino acid sequence of the antimicrobial peptide P16V is shown in SEQ ID NO: 6.

In another aspect, the disclosure provides a method for preparing the antimicrobial peptide; the method employs a solid-phase synthesis technique.

Furthermore, the disclosure provides a method for prevention or treatment of a scalp disease caused by dysbiosis of scalp microbiome, the method comprising: administering to a patient in need thereof a product comprising a therapeutically effective amount of the aforementioned antimicrobial peptide.

Optionally, the product is selected from the group consisting of scalp care products, hair conditioners, shampoos, and topical medications.

The following provides a detailed explanation of the above technical solutions, accompanied by examples.

The experimental methods used in the following examples are conventional methods unless otherwise stated. The materials, reagents, and other components used in the following examples can be obtained from commercial sources.

### Example 1

The disclosure provides an antimicrobial peptide P16 for maintaining the health of the scalp microbiome. The antimicrobial peptide P16 has an amino acid sequence shown in SEQ ID NO: 1: KWLKRIKKLFAN.

The disclosure further provides a method for preparing the antimicrobial peptide; the method employs a solid-phase synthesis technique as follows:

(1) The antimicrobial peptide was synthesized from C-terminus to N-terminus: 20 g of AM resin was placed in a reaction tube, and 15 mL/g of dichloromethane (DCM) was added to the reaction tube; the reaction tube was shaken for 45 minutes; the solvent DCM was filtered off by a sintered glass funnel from the reaction tube, and a 3 equivalents (3x molar excess) of Fmoc-Linker-OH (CAS: 145069-56-3) was added, followed by a 6.5 equivalents (3× molar excess) of N,N-diisopropylethylamine (DIEA). The reaction tube was shaken for 75 minutes. The reaction was quenched with methanol; the DMF was removed by rotary evaporation; 20 mL/g 20% (V/V) piperidine-DMF solvent was added for washing for 5 minutes, then removed, and 20 mL/g 20% (V/V) piperidine DMF solvent was added for washing for 20 minutes. The resin was washed sequentially with DMF (15 mL/g), DCM (15 mL/g), and DMF (15 mL/g) for two times, and shaken for 40 minutes.

(2) For the coupling of the first amino acid: the solvent DCM was filtered off through a sintered glass funnel. 3.5 equivalents of Fmoc-Ala-OH were added and dissolved in DMF, followed by the addition of 6.5 equivalents of N,N-diisopropylethylamine (DIEA). The mixture was shaken for 70 minutes and quenched with methanol.

(3) Deprotection: DMF was removed by rotary evaporation. 20% (v/v) piperidine-DMF solution (20 mL/g) was added for washing for 5 minutes and then removed. Subsequently, a 20% (v/v) piperidine-DMF solution (15 mL/g) was added for washing for 20 minutes.

(4) Testing: the piperidine solution was removed. Fifteen resin beads were collected and washed three times with ethanol, followed by the addition of a detection reagent. The mixture was heated at 105-110° C. for 8 minutes. If the color of the mixture turned dark blue, a positive reaction was confirmed.

(5) Rinse resin: the resin was rinsed sequentially with DMF (15 mL/g), DCM (15 mL/g), and DMF (15 mL/g) twice.

(6) Condensation: 3 equivalents of the next amino acid derivative (from C-to N-terminus) dissolved in DMF and 3.5 equivalents of HBTU (O-Benzotriazole-N,N,N′,N′-tetramethyluronium hexafluorophosphate) dissolved in DMF were added to the reaction tube. Thereafter, 16 equivalents of DIEA (N,N-Diisopropylethylamine) was added. The reaction was allowed to proceed for 40 minutes.

(7) Testing: fifteen resin beads were collected and washed three times with ethanol, and then a detection reagent was added. The mixture was heated at 105-110° C. for 5 minutes. If the mixture was colorless, a negative reaction was confirmed.

(8) Rinse resin: the resin was rinsed sequentially with DMF (10 mL/g), DCM (10 mL/g), and DMF (10 mL/g) twice.

(9) The operations (3) through (8) were repeated to sequentially couple the amino acids as shown in SEQ ID NO: 1 in a right-to-left order.

(10) The resin was drained for 10 minutes and subjected to sequential washing as follows: twice with DMF (15 mL/g), twice with methanol (15 mL/g), twice with DMF (15 mL/g), and twice with DCM (15 mL/g).

(11) Cleavage of the peptide from the resin: the cleavage cocktail used at 25° C. consisted of 95% (v/v) TFA (trifluoroacetic acid), 1% (v/v) water, 2% (v/v) EDT (β-mercaptoethanol), and 2% (v/v) TIS (triisopropylsilane). The cleavage reaction was carried out for 120 minutes, to yield a cleavage solution.

(12) Blow-drying and washing: the cleavage solution was blown dry with nitrogen and washed six times with ether, and then allowed to evaporate at room temperature.

(13) Analytical purification and lyophilization: the crude peptide was purified by high-performance liquid chromatography (HPLC); the peptide solution was collected and concentrated in a lyophilizer to yield a white powder, which was an antimicrobial peptide with a sequence of KWLKRIKKLFAN.

The detection reagent used was ninhydrin.

### Example 2

The disclosure provides an antimicrobial peptide P16L for maintaining the health of the scalp microbiome. The antimicrobial peptide P16L has an amino acid sequence shown in SEQ ID NO: 2: KWLKRIKKLFLN.

P16L's variants are listed below:

| Variants | Sequence ID |
|---|---|
| K1A | AWLKRIKKLFLN (SEQ ID NO: 7) |
| W2A | KALKRIKKLFLN (SEQ ID NO: 8) |
| L3A | KWAKRIKKLFLN (SEQ ID NO: 9) |
| K4A | KWLARIKKLFLN (SEQ ID NO: 10) |
| R5A | KWLKAIKKLFLN (SEQ ID NO: 11) |
| I6A | KWLKRAKKLFLN (SEQ ID NO: 12) |
| K7A | KWLKRIAKLFLN (SEQ ID NO: 13) |
| K8A | KWLKRIKALFLN (SEQ ID NO: 14) |
| L9A | KWLKRIKKAFLN (SEQ ID NO: 15) |
| F10A | KWLKRIKKLALN (SEQ ID NO: 16) |
| N12A | KWLKRIKKLFLA (SEQ ID NO: 17) |

### Example 3

The disclosure provides an antimicrobial peptide P16K for maintaining the health of the scalp microbiome. The antimicrobial peptide P16K has an amino acid sequence shown in SEQ ID NO: 3: KWLKRIKKLFKN.

### Example 4

The disclosure provides an antimicrobial peptide P16R for maintaining the health of the scalp microbiome. The antimicrobial peptide P16R has an amino acid sequence shown in SEQ ID NO: 4: KWLKRIKKLFRN.

### Example 5

The disclosure provides an antimicrobial peptide P16I for maintaining the health of the scalp microbiome. The antimicrobial peptide P16I has an amino acid sequence shown in SEQ ID NO: 5: KWLKRIKKLFIN.

### Example 6

The disclosure provides an antimicrobial peptide P16V for maintaining the health of the scalp microbiome. The antimicrobial peptide P16V has an amino acid sequence shown in SEQ ID NO: 6: KWLKRIKKLFVN.

### EXPERIMENTAL EXAMPLES

### 1. Antimicrobial Test

(1) The antibacterial activity assay of the antimicrobial peptides described in Examples 1-6 in liquid culture media was conducted. The tested bacterial and fungal strains included: Malassezia furfur ATCC 44344; Malassezia restricta ATCC 33081; Staphylococcus hominis ATCC 23976; Candida albicans ATCC 14053; Staphylococcus aureus ATCC 25913; and Lactobacillus acidophilus ATCC 4356. The control groups included chloramphenicol (CAP) and ketoconazole (KCZ), both of which were commercially available.

### (2) Experimental Results

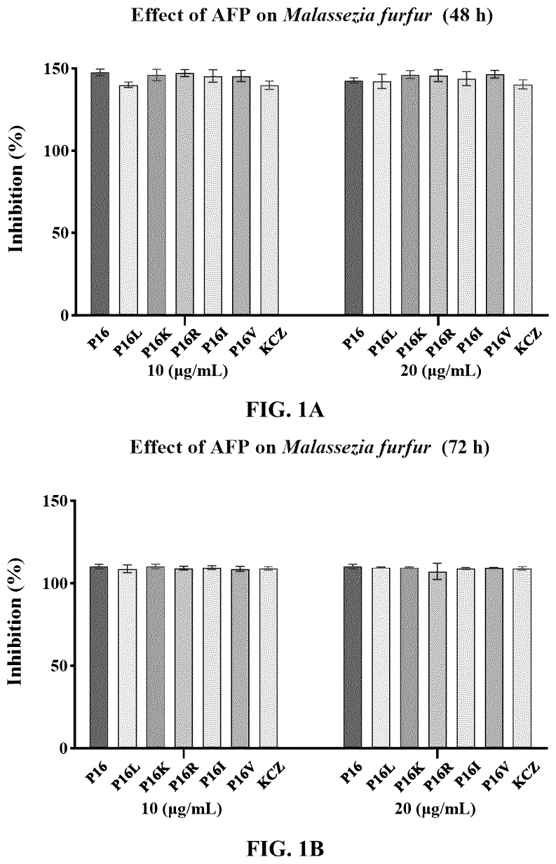

The results of the antibacterial experiments on Malassezia furfur are shown in FIGS. 1A and 1B. The inhibition rate of all the antimicrobial peptides against Malassezia furfur reached 100% after 48 and 72 hours of incubation of the bacteria with 10 μg/mL of the antimicrobial peptides. Similarly, all the antimicrobial peptides showed 100% inhibition against Malassezia furfur after incubation of the bacteria with 20 μg/mL of the antimicrobial peptides for 48 hours and 72 hours.

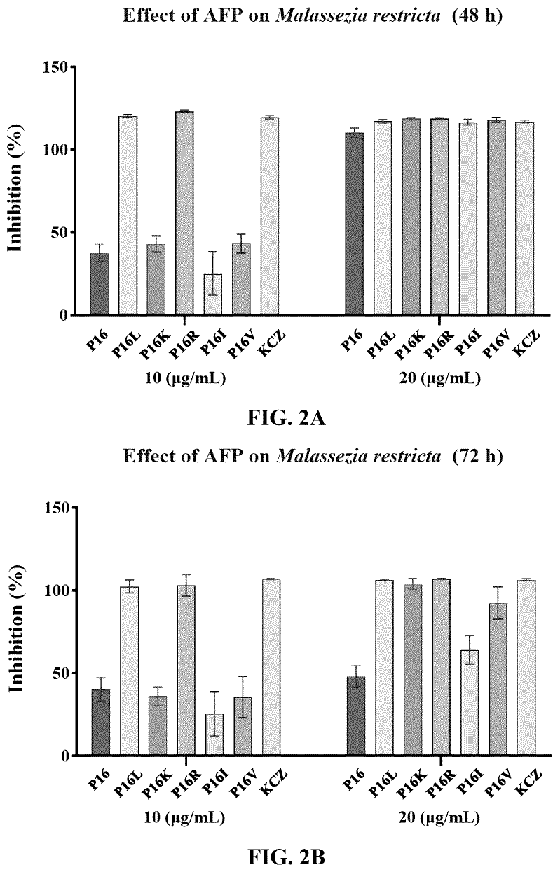

The results of the anti-fungal experiments on Malassezia restricta are shown in FIGS. 2A and 2B. After incubation of the bacteria with 10 μg/mL of the antimicrobial peptides for 48 hours, the inhibition rate of the antimicrobial peptides P16L and P16R against Malassezia restricta reached 100%. After continuous incubation for 72 hours, the inhibition rate of P16L and P16R against Malassezia restricta remained unchanged, at 100%. Similarly, after incubation of the bacteria with 20 μg/mL of the antimicrobial peptides for 48 hours, the inhibition rates of the antimicrobial peptides P16, P16L, P16K, P16R, P16I, and P16V against Malassezia restricta reached 100%. After continuous incubation for 72 hours, the antibacterial peptides P16L, P16K, and P16R maintained a 100% inhibition rate against Malassezia restricta.

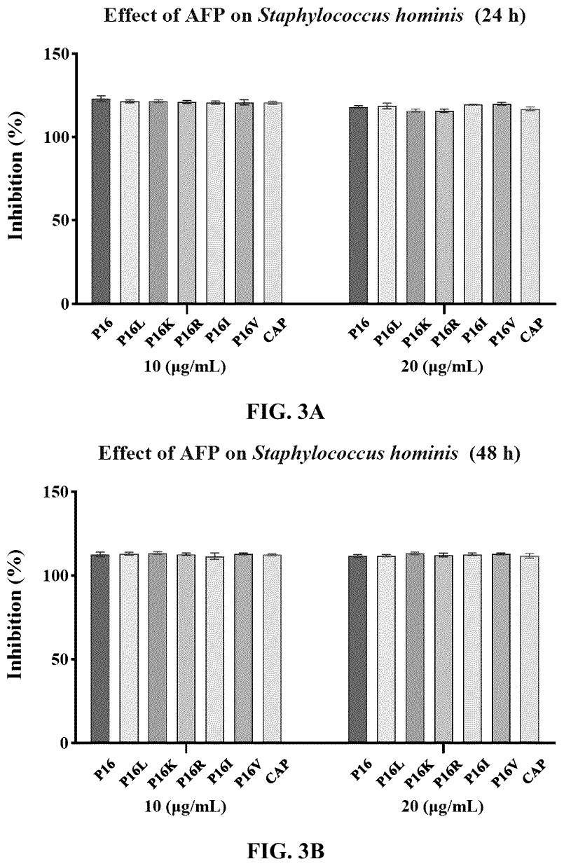

The results of the antibacterial experiments on Staphylococcus hominis are shown in FIGS. 3A and 3B. After incubation of the bacteria with 10 μg/mL and 20 μg/mL of the antimicrobial peptides for 24 hours, the inhibition rates of all antimicrobial peptides against Staphylococcus hominis reached 100%. After continuous incubation for 48 hours, the inhibition rates of all antimicrobial peptides against Staphylococcus hominis reached 100%.

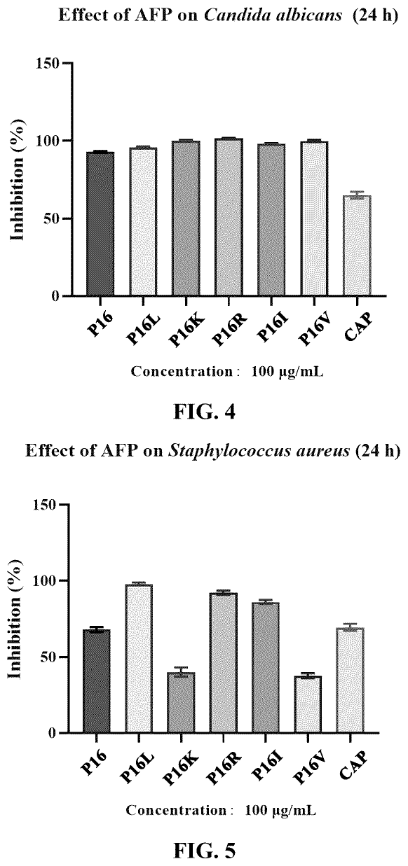

The results of the anti-fungal experiments on Candida albicans were shown in FIG. 4. After incubation of the bacteria with 100 μg/mL of the antimicrobial peptides for 24 hours, the inhibition rates of the antimicrobial peptides P16, P16L, P16K, P16R, P16I, and P16V against Candida albicans reached 100%.

The results of the antibacterial experiments on Staphylococcus aureus were shown in FIG. 5. After incubation of the bacteria with 100 μg/mL of the antimicrobial peptides for 24 hours, the inhibition rates of the antimicrobial peptides P16L, P16R, and P16I against Staphylococcus aureus reached 100%.

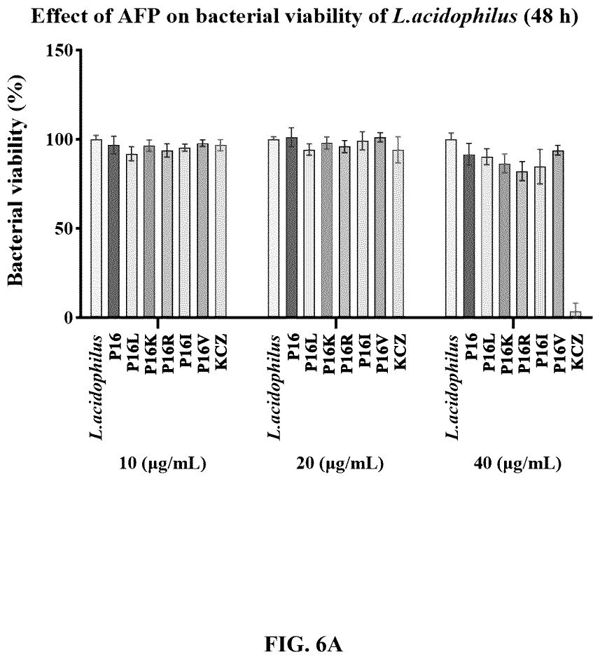

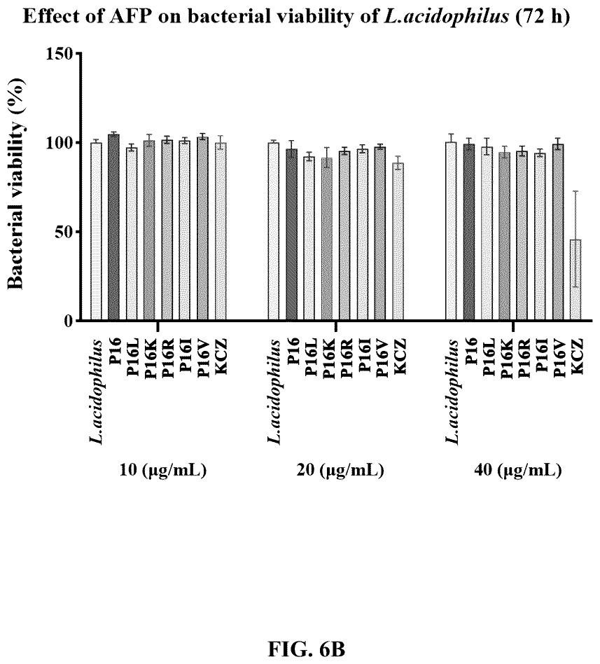

The results of the antibacterial experiments on Lactobacillus acidophilus are shown in FIGS. 6A and 6B. After incubation of the bacteria with 10 μg/mL, 20 μg/mL, and 40 μg/mL of the antimicrobial peptides for 48 and 72 hours, the inhibition rates of all antimicrobial peptides against Lactobacillus acidophilus were less than 20%.

### Anti-Fungal Tests

Malassezia restricta (CICC 33081) were inoculated into liquid BHI (qdrishui 10805) medium and incubated at 37° C. for 24 or 48 hours. The cultures were then diluted several-fold with sterile water to prepare fungal suspensions. A 20 μL aliquot of the suspension was added to 200 μL of the test reagent (containing the peptides of P16L variants, positive or negative controls), mixed thoroughly, and incubated at room temperature for 2 minutes. Subsequently, the mixture was spread evenly onto pre-prepared solid BHI agar plates. After incubation at 37° C. for 24 or 48 hours, colony formation was observed, photographed, and counted.

### Results:

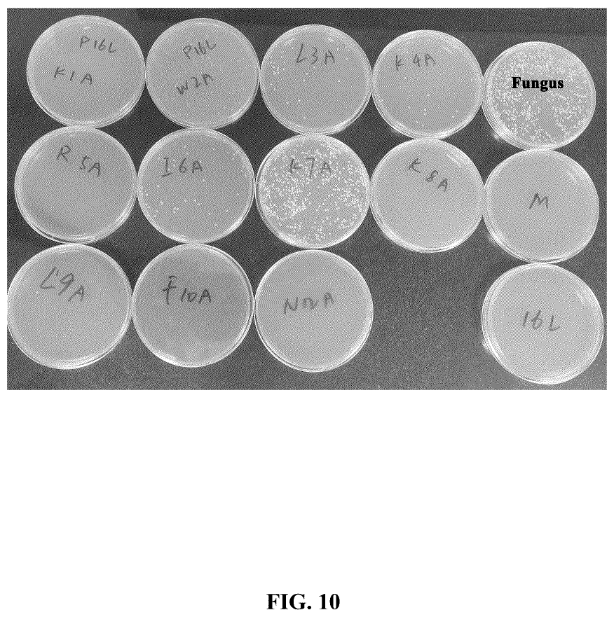

As shown in FIG. 10, 10 ppm of P16L variants KIA, R5A, K8A, L9A, F10A, N12A completely inhibited the growth of Malassezia restricta; 10 ppm of P16L variants L3A, K4A, 16A partially inhibited the growth of Malassezia restricta. This means the motif that contributes to the anti-fungal effect of P16L is X₁-W-L-K-X₅-I-K-X₈-X₉-X₁₀-L-X₁₂ (SEQ ID NO: 18).

### 2. Anti-Inflammatory Experiments

In vitro anti-inflammatory cell experiments of the antimicrobial peptides described in Examples 1-6 were conducted.

### (1) Experimental Methods and Materials

The murine macrophage cell line RAW264.7 (ATCC No. TIB-71) was cultured in DMEM medium supplemented with 10% fetal bovine serum (FBS) under standard conditions (37° C., 5% CO₂). The cells were seeded into a 96-well plate at a density of 1×10⁵ cells/well and pre-cultured for 24 hours. The cells were stimulated with lipopolysaccharide (LPS, 10 μg/mL) for 24 hours, to establish an in vitro inflammation model. Concurrent with LPS stimulation, the antimicrobial peptides from Examples 1-6 (20 ug/mL) were added to designated groups, with three replicates per group. The experimental groups included: a blank control group (no antimicrobial peptides or LPS), a model control group (LPS stimulation only), and a positive control group (dexamethasone, DEX, 20 μg/mL). The anti-inflammatory activity of the antimicrobial peptides was evaluated by measuring the levels of the inflammatory mediators NO (detected via the Griess assay) and IL-1β/IL-6 (quantified using ELISA kits from R&D Systems) in the cell supernatants. The experimental data are presented as Mean±SD. Statistical analysis was performed using one-way ANOVA in GraphPad Prism 9.0, with significance thresholds defined as ***P<0.001, *P<0.01, and P<0.05.

### (2) Experimental Results

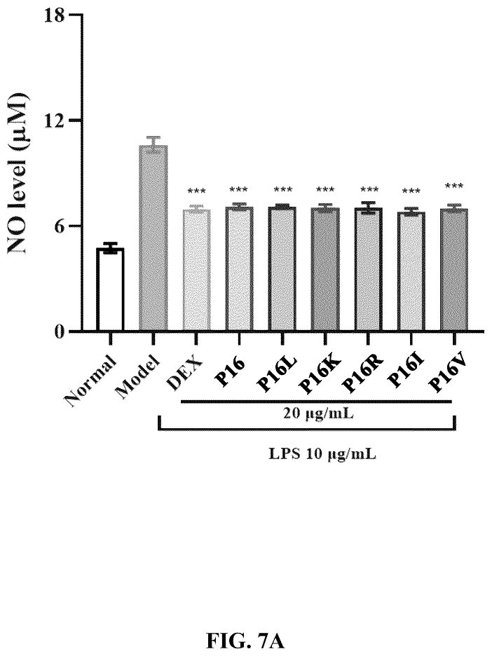

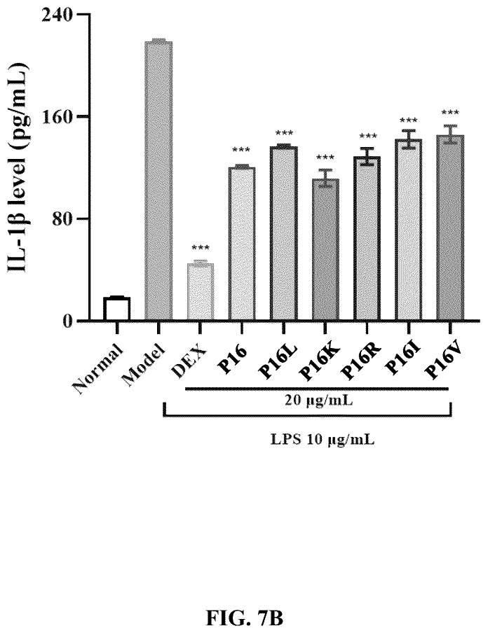

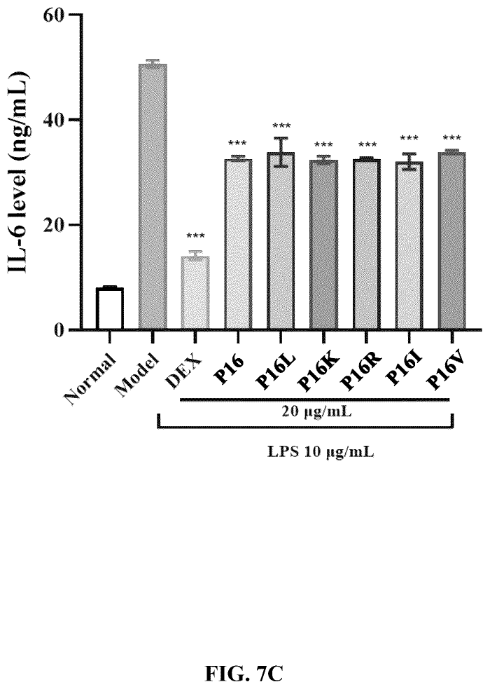

The LPS-induced anti-inflammatory effects are shown in FIGS. 7A, 7B, and 7C. At a concentration of 20 μg/mL, the antimicrobial peptides P16, P16L, P16K, P16R, P16I, and P16V significantly inhibited the LPS-induced release of NO, IL-1β, and IL-6, demonstrating potent anti-inflammatory activity.

### 3. Anti-Allergy Experiment

In vitro anti-allergic cell experiments of the antimicrobial peptides described in Examples 1-6 were conducted.

### (1) Experimental Methods and Materials

The murine mastocytoma cell line P815 (ECACC No. 86021901) was cultured in RPMI-1640 medium supplemented with 10% fetal bovine serum (FBS) under standard conditions (37° C., 5% CO₂). When the cells reached the logarithmic growth phase, they were seeded into a 96-well plate at a density of 1×10⁵ cells/well and pre-cultured for 24 hours. The original medium was then replaced with medium containing substance P (SP, 0.1 μM) to stimulate mast cell degranulation for 24 hours. Concurrent with SP stimulation, the antimicrobial peptides of Examples 1-6 (20 μg/mL) were added to the designated groups, with three replicates per group. The experimental groups included a blank control group (no SP or antimicrobial peptides), a model control group (SP stimulation only), and a positive control group (sodium cromoglicate, 20 μg/mL). The supernatants were collected to measure β-hexosaminidase activity in cell lysates (using the p-nitrophenyl-N-acetyl-β-D-glucosaminide substrate method, reflecting degranulation levels) and IL-1β/IL-6 concentrations (via R&D Systems ELISA kits) to evaluate the anti-allergic and anti-inflammatory activity of the antimicrobial peptides. The experimental data are presented as Mean±SD and analyzed using one-way ANOVA in GraphPad Prism 9.0, with significance levels defined as ***P<0.001, *P: 0.01, and P<0.05.

### (2) Experimental Results

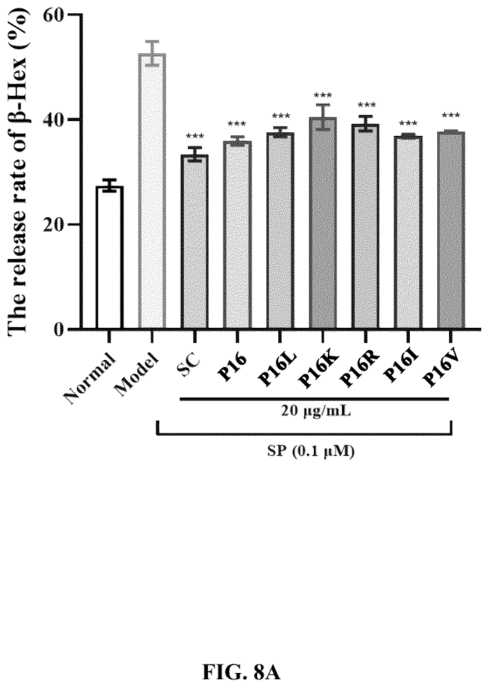

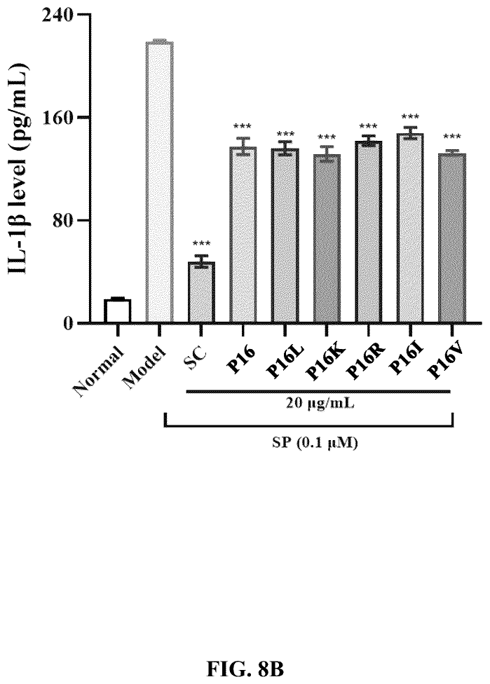

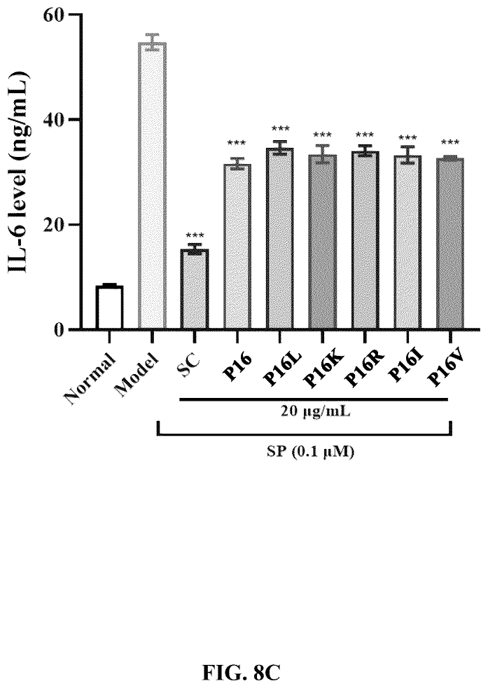

The results of the substance P (SP)-induced anti-allergic and anti-inflammatory effects are shown in FIGS. 8A, 8B, and 8C. At the concentration of 20 μg/mL, the antimicrobial peptides P16, P16L, P16K, P16R, P16I, and P16V significantly inhibited SP-induced release of β-hexosaminidase, IL-1β, and IL-6, demonstrating potent anti-allergic and anti-inflammatory effects.

### 4. Human Dandruff Removal Experiment

Human clinical trials of the antimicrobial peptide in Example 2 were conducted.

### (1) Experimental Methods

Study population: 66 volunteers aged between 18 and 55 years.

Scalp condition: all volunteers exhibited mild to moderate dandruff and scalp itching.

Exclusion criteria: known allergy to shampoo ingredients, pre-existing dermatological conditions, or pregnancy/lactation.

### Grouping and Treatment:

Treatment group (33 volunteers): scalp serum containing 0.2% P16L.

Control group (33 volunteers): base formula without P16L.

Application: in the treatment group, the serum with P16L was applied to the entire scalp twice a day, and in the control group, the serum without P16L was applied to the entire scalp twice a day.

Evaluation metrics (assessed at baseline, day 14, and day 28 of treatment):

Desquamation index: clinical scoring on a 0-10 scale (0=no visible scaling, 10=severe scaling).

Scalp hydration: measured via the capacitance method using Cornetometer® CM825.

Scalp barrier function: quantified by transepidermal water loss (TEWL, g/m²/h) using Tewameter® TM300.

### (2) Experimental Results

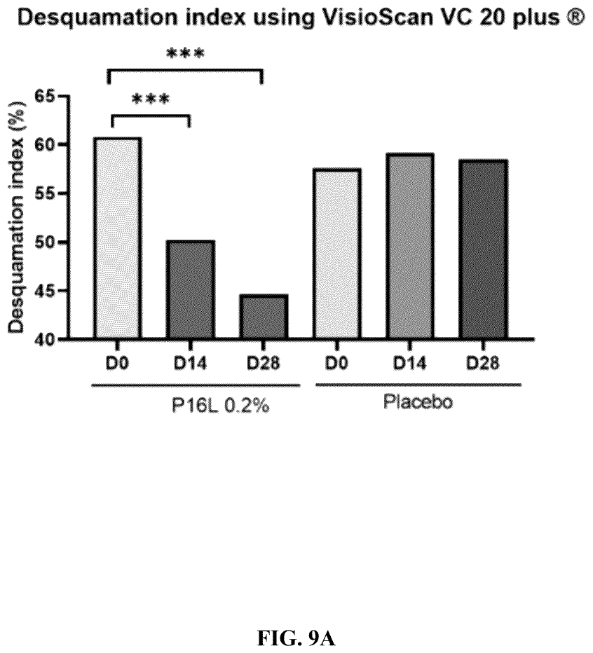

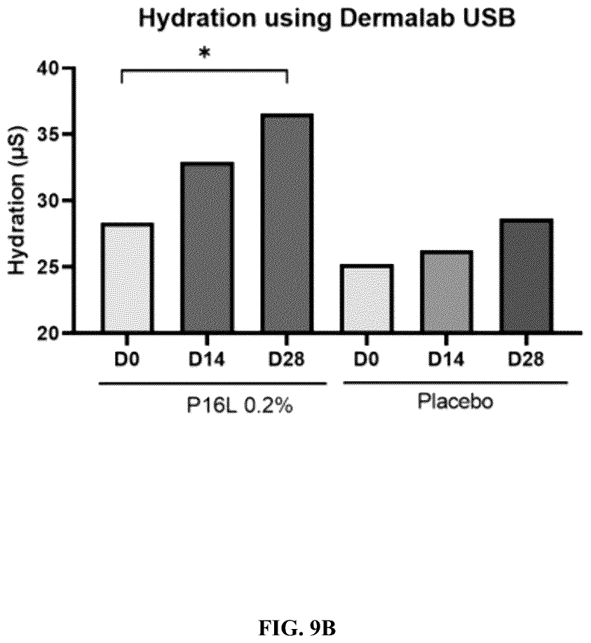

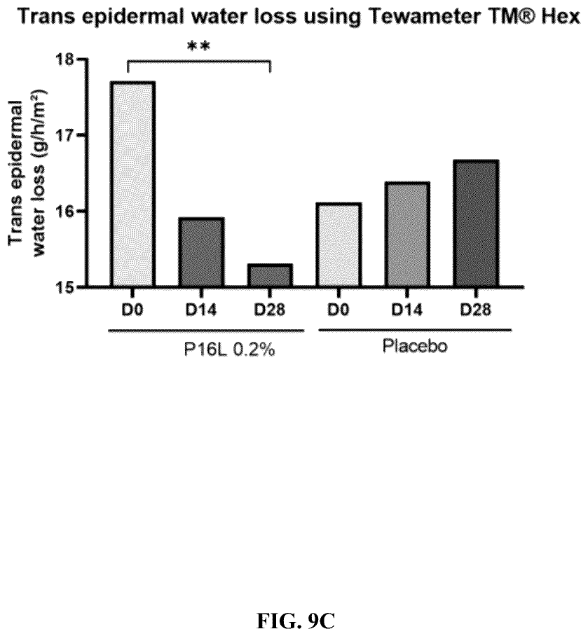

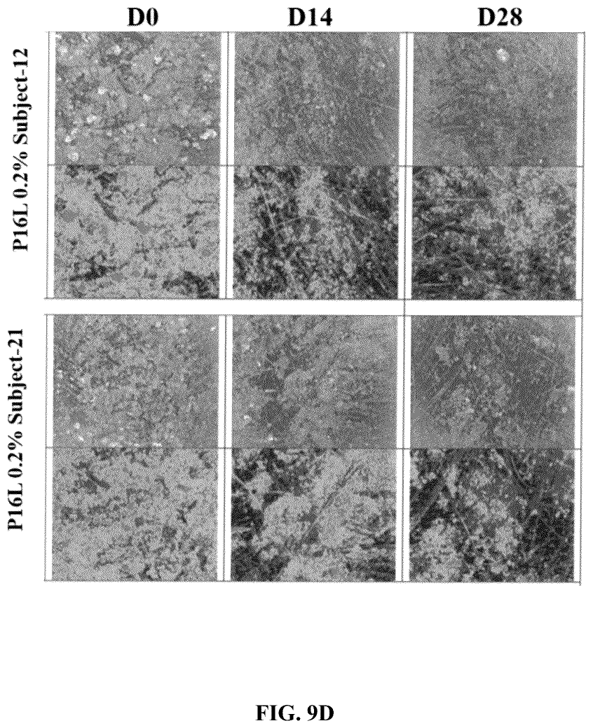

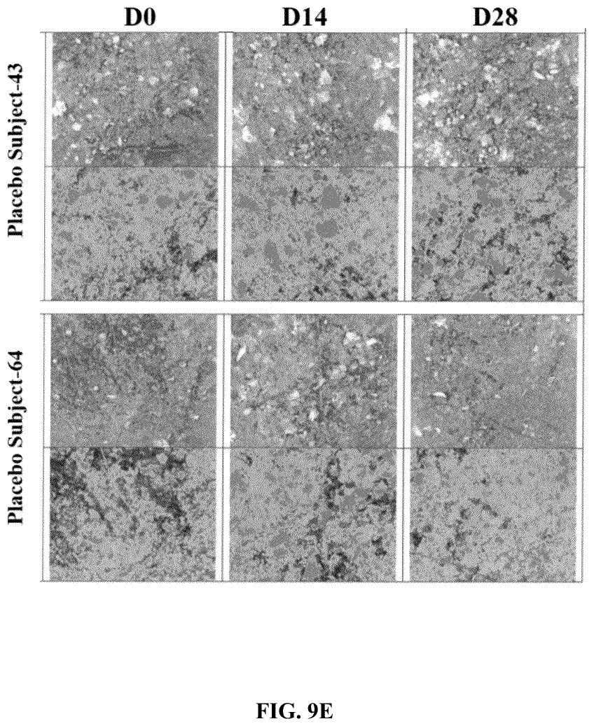

The results of the human trial are shown in FIGS. 9A, 9B, 9C, 9D and 9E; the antimicrobial peptide P16 of the disclosure is more advantageous compared to its counterpart. The incidence of adverse reaction events in the treatment group was not significantly different from that in the placebo group, with no contact dermatitis or microbial resistance reported.

The antimicrobial peptide for maintaining the health of the scalp microbiome of the disclosure exhibits excellent safety for treating scalp conditions caused by microbial dysbiosis, including dandruff, seborrheic dermatitis, scalp pruritus, and hair loss. The antimicrobial peptide can be incorporated into anti-dandruff or therapeutic formulations such as scalp care products, conditioners, shampoos, or topical pharmaceuticals targeting seborrheic dermatitis, pruritus, or alopecia. Notably, the antimicrobial peptide demonstrates significant inhibitory effects against pathogenic microbes including Malassezia furfur, Malassezia restricta, Staphylococcus hominis, Candida albicans, and Staphylococcus aureus, while preserving the growth of beneficial bacteria (e.g., Lactobacillus acidophilus). This selective antimicrobial activity promotes scalp microbiome homeostasis, highlighting its substantial therapeutic and commercial potential.

It will be obvious to those skilled in the art that changes and modifications may be made, and therefore, the aim in the appended claims is to cover all such changes and modifications.

What is claimed is:

1. An antimicrobial peptide, comprising one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide resulting from substitution, deletion, and/or addition of one or more amino acid residues of the polypeptide in (1) or (2);
(4) a polypeptide obtained by modifying a N-terminus and/or a C-terminus of the polypeptide in (1) or (2); and
(5) a polypeptide having at least 85%, 90%, 95%, 98%, or 99% sequence identity to the amino acid sequence of the polypeptide in (1) or (2).

2. The antimicrobial peptide of claim 1, wherein the antimicrobial peptide is selected from P16, P16L, P16K, P16R, P16I, or P16V.

3. The antimicrobial peptide of claim 2, wherein the amino acid sequence of the antimicrobial peptide P16 is shown in SEQ ID NO: 1; the amino acid sequence of the antimicrobial peptide P16L is shown in SEQ ID NO: 2; and the amino acid sequence of the antimicrobial peptide P16K is shown in SEQ ID NO: 3.

4. The antimicrobial peptide of claim 2, wherein the amino acid sequence of the antimicrobial peptide P16R is shown in SEQ ID NO: 4; the amino acid sequence of the antimicrobial peptide P16I is shown in SEQ ID NO: 5; and the amino acid sequence of the antimicrobial peptide P16V is shown in SEQ ID NO: 6.

5. The antimicrobial peptide of claim 2, wherein the amino acid sequence of the antimicrobial peptide P16L has a motif X-W-L-K-X-I-K-X-X-X-L-X (SEQ ID NO: 18) for anti-fungal effect.

6. A method for preparing the antimicrobial peptide of claim 1, the method employing a solid-phase synthesis technique.

7. A method for prevention or treatment of a scalp disease caused by dysbiosis of scalp microbiome, the method comprising: administering to a patient in need thereof a product comprising a therapeutically effective amount of an antimicrobial peptide of claim 1.

8. The method of claim 7, wherein the product is selected from the group consisting of scalp care products, hair conditioners, shampoos, and topical medications.


SEQUENCE LISTING (18 sequences)

SEQ ID NO: 1 - protein, 12 aa, synthetic construct
KWLKRIKKLFAN

SEQ ID NO: 2 - protein, 12 aa, synthetic construct
KWLKRIKKLFLN

SEQ ID NO: 3 - protein, 12 aa, synthetic construct
KWLKRIKKLFKN

SEQ ID NO: 4 - protein, 12 aa, synthetic construct
KWLKRIKKLFRN

SEQ ID NO: 5 - protein, 12 aa, synthetic construct
KWLKRIKKLFIN

SEQ ID NO: 6 - protein, 12 aa, synthetic construct
KWLKRIKKLFVN

SEQ ID NO: 7 - protein, 12 aa, synthetic construct
AWLKRIKKLFLN

SEQ ID NO: 8 - protein, 12 aa, synthetic construct
KALKRIKKLFLN

SEQ ID NO: 9 - protein, 12 aa, synthetic construct
KWAKRIKKLFLN

SEQ ID NO: 10 - protein, 12 aa, synthetic construct
KWLARIKKLFLN

SEQ ID NO: 11 - protein, 12 aa, synthetic construct
KWLKAIKKLFLN

SEQ ID NO: 12 - protein, 12 aa, synthetic construct
KWLKRAKKLFLN

SEQ ID NO: 13 - protein, 12 aa, synthetic construct
KWLKRIAKLFLN

SEQ ID NO: 14 - protein, 12 aa, synthetic construct
KWLKRIKALFLN

SEQ ID NO: 15 - protein, 12 aa, synthetic construct
KWLKRIKKAFLN

SEQ ID NO: 16 - protein, 12 aa, synthetic construct
KWLKRIKKLALN

SEQ ID NO: 17 - protein, 12 aa, synthetic construct
KWLKRIKKLFLA

SEQ ID NO: 18 - protein, 12 aa, synthetic construct
XWLKXIKXXXLX